(19) United States
(12) Patent Application Publication
(10) Pub. No.: US 2014/0288447 A1
(43) Pub. Date: Sep. 25, 2014

(54) EAR-RELATED DEVICES IMPLEMENTING SENSORS TO ACQUIRE PHYSIOLOGICAL CHARACTERISTICS

(71) Applicants: Michael Edward Smith Luna, San Jose, CA (US); Thomas Alan Donaldson, Nailsworth (GB); Scott Fullam, Palo Alto, CA (US)

(72) Inventors: Michael Edward Smith Luna, San Jose, CA (US); Thomas Alan Donaldson, Nailsworth (GB); Scott Fullam, Palo Alto, CA (US)

(73) Assignee: AliphCom, San Francisco, CA (US)

(21) Appl. No.: 14/209,690

(22) Filed: Mar. 13, 2014

Related U.S. Application Data
(60) Provisional application No. 61/785,743, filed on Mar. 14, 2013.

(51) Int. Cl. A61B 5/024; A61B 5/0245

(52) U.S. Cl. CPC A61B 5/02438; A61B 5/0245
USPC 600/508

(57) ABSTRACT

Various embodiments relate generally to electrical and electronic hardware, computer software, wired and wireless network communications, and wearable computing and audio devices for monitoring health and wellness. More specifically, disclosed are an apparatus and a method for processing signals representing physiological characteristics sensed from tissue at or adjacent an ear of an organism. In one or more embodiments, a wearable device includes one or more sensor terminals, one or more physiological sensors configured to sense one or more signals originating at the one or more sensor terminals. At least one sensor terminal includes a pressure-sensitive terminal configured to detect a pressure exerted by a portion of tissue of an organism and generate a pressure signal representing a value of the pressure. Further, the wearable device can include a processor configured to cause generation of data representing a physiological characteristic of the organism based on the pressure signal.

### CROSS-REFERENCE TO RELATED APPLICATIONS

This application is a U.S. non-provisional patent application that claims the benefit of U.S. Provisional Patent Application No. 61/785,743, filed Mar. 14, 2013, and entitled “SENSING PHYSIOLOGICAL CHARACTERISTICS IN ASSOCIATION WITH EAR-RELATED DEVICES OR IMPLEMENTS,” which is herein incorporated by reference for all purposes.

### FIELD

Various embodiments relate generally to electrical and electronic hardware, computer software, wired and wireless network communications, and wearable computing and audio devices for monitoring health and wellness. More specifically, disclosed are an apparatus and a method for processing signals representing physiological characteristics sensed from tissue at or adjacent an ear of an organism.

### BACKGROUND

Conventional techniques for acquiring physiological information from an organism, such as a human, typically required the assistance of trained medical personnel. While there exists some devices and techniques for a layperson to determine physiological characteristics, such as heart rate, such devices are not well-suited for everyday activities of active people. Typical devices for determining physiological characteristics are typically designed to attach to a proximal portion of a limb, such as an upper arm, or about or on the chest of the user.

Thus, what is needed is a solution for data capture devices, such as for wearable devices, without the limitations of conventional techniques.

### BRIEF DESCRIPTION OF THE DRAWINGS

Various embodiments or examples (“examples”) of the invention are disclosed in the following detailed description and the accompanying drawings:

FIG. 1A illustrates an example of an implementation of the ear-related device/implement configured to facilitate sensing of physiological signals, according to some embodiments;

FIG. 1B is a diagram illustrating another example of an ear-related device/implement, according to some embodiments;

FIG. 2 depicts an ear-related device/implement configured to receive signals describing physiological characteristics, according to some embodiments;

FIG. 3A depicts another example of an ear-related device/implement configured to provide for sensor terminals to sense physiological characteristics, according to some embodiments;

FIG. 3B depicts a variation of yet another example of an ear-related device/implement configured to provide for sensor terminals to sense physiological characteristics, according to some embodiments;

FIG. 4A depicts perspective and top views of the ear-related device/implement shown in FIG. 3A, according to some examples;

FIG. 4B depicts perspective and top views of yet another example of an ear-related device/implement shown in FIG. 4A, according to some examples;

FIG. 4C depicts an example of the coupling of an ear-related device shown in FIG. 4B to an ear, according to some examples;

FIG. 5 depicts another example of an ear-related device/implement configured to provide for sensor terminals to sense physiological characteristics, according to some embodiments;

FIG. 6 depicts another example of an ear-related device/implement configured to provide for sensor terminals to sense physiological characteristics, according to some embodiments;

FIG. 7 depicts another example of an ear-related device/implement configured to provide sensor terminals to sense physiological characteristics, according to some embodiments;

FIG. 8 depicts yet another example of an ear-related device/implement configured to provide sensor terminals to sense physiological characteristics, according some embodiments;

FIG. 9 is a diagram depicting a sensor controller, according to some examples;

FIG. 10 is a diagram depicting an ear device implementing an optical sensor, according to some examples;

FIG. 11 is a diagram depicting an ear device implementing an environmental sensor, according to some examples; and

FIG. 12 illustrates an exemplary computing platform disposed in or otherwise associated with an ear-related device/implement in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

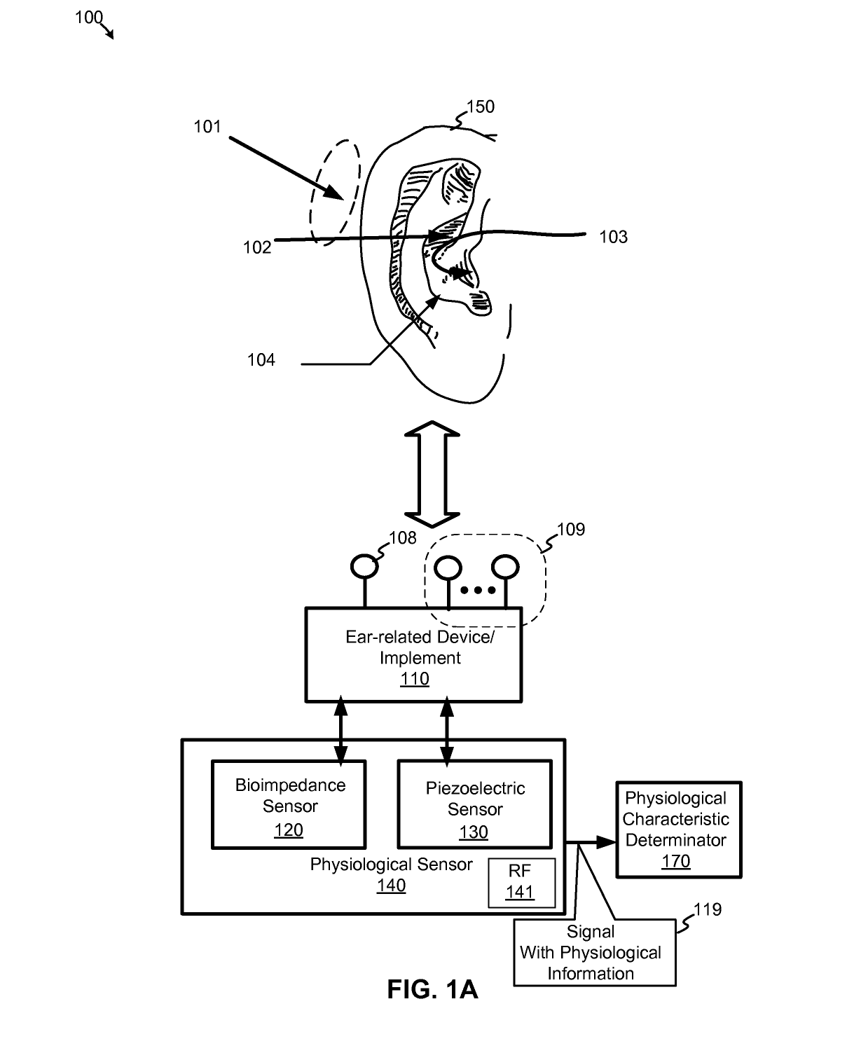

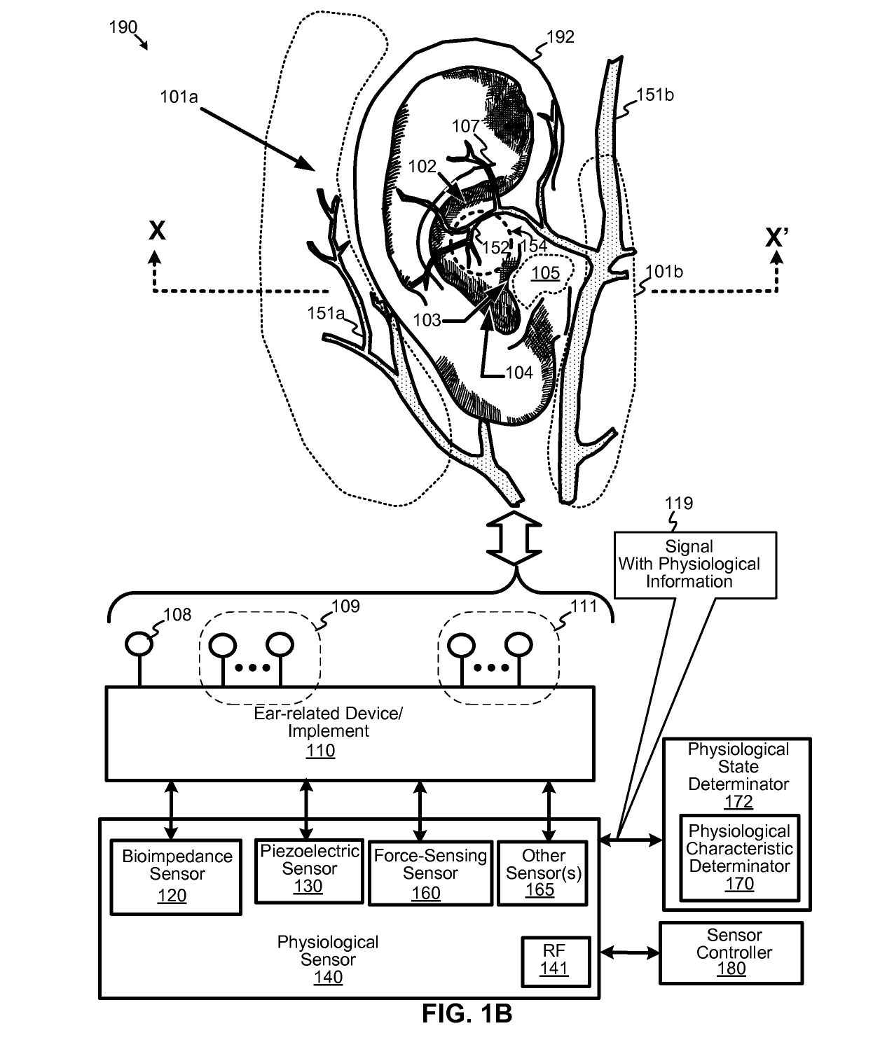

FIG. 1A illustrates an example of an implementation of the ear related device/implement configured to facilitate sensing of physiological signals, according to some embodiments. Diagram 100 depicts an ear-related device/implement 110 coupled to at least one sensor terminal 108. In some examples, ear-related device/implement 110 can be coupled to multiple sensor terminals including sensor terminal 108 and any number of sensor terminals 109. As shown, any of the sensor terminals 108 and 109 can be positioned to sense physiological signals from or at various portions of tissue at or near ear 150 or regions thereabout, such as a region of skin 101, which is behind the ear 150. In one example, a sensor terminal can be positioned adjacent a concha 102 portion of ear 150 (e.g., a cymba concha portion of the ear). In another example, a sensor terminal can be positioned adjacent a portion of a targus region 103 of ear 150. In still another example, a sensor terminal can be position adjacent to a portion of another concha portion or region 104 of ear 150 (e.g., cavum concha portion of the ear). Also, the sensor terminal can be disposed adjacent a region of tissue 101. Sensor terminals 108 and are not limited to sensing physiological signals from the above-identified regions, but rather can sense physiological signals from any part of ear 150. Examples of ear-related device/implement 110 include headsets (e.g., Bluetooth® headsets), headphones (e.g., wireless headphones), and any other device. For example, ear-related device/implement 110 can include or be disposed in the speaker portion of a mobile computing device or mobile phone, or any other device configured to, for instance, provide audio or facilitate in uni- or bi-directional communications. In some cases, ear-related device/implement 110 can include implements such as eyewear (e.g., including the portions that extend behind an ear), hats (e.g., including those portions that extend behind or over an ear), earbuds, or any other instrument or implement upon which at least sensor terminals can be disposed.

Diagram 100 depicts a physiological sensor 140 configured to generate one or more physiological signals that can be used to derive physiological signals, such as heart rate, respiration, and other detectable physiological characteristics, for example, from the sensor terminals. Any ear-related device can include a physiological sensor 140 and a physiological characteristic determinator 170, which can be implemented as a physiological signal generator in some embodiments. Physiological sensor 140 can be configured to sense signals, such as physiological signals, associated with a physiological characteristic.

Ear-related device/implement 110 can coupled to or can include a physiological sensor 140 and/or a physiological characteristic determinator 170. Physiological sensor 140 is configured to receive the sensed signals from one or more of the sensor terminal 108 and/or any of sensor terminals 109. In one embodiment, physiological sensor 140 includes a bioimpedance sensor 120. In some embodiments, sensor terminal 108 and/or any of sensor terminals 109 are electrodes coupled to bioimpedance sensor 120, which is configured to determine the bioelectric impedance (“bioimpedance”) of one or more types of tissues of a wearer to identify, measure, and monitor physiological characteristics. For example, a drive signal having a known amplitude and frequency can be applied to a user, from which a sink signal is received as bioimpedance signal. The bioimpedance signal is a measured signal that includes real and complex components. Examples of real components include extra-cellular and intra-cellular spaces of tissue, among other things, and examples of complex components include cellular membrane capacitance, among other things. Further, the measured bioimpedance signal can include real and/or complex components associated with arterial structures (e.g., arterial cells, etc.) and the presence (or absence) of blood pulsing through an arterial structure. In some examples, a heart rate signal, or other physiological signals, can be determined (i.e., recovered) from the measured bioimpedance signal by, for example, comparing the measured bioimpedance signal against the waveform of the drive signal to determine a phase delay (or shift) of the measured complex components. The bioimpedance sensor signals can provide a heart rate, a respiration rate, and a Mayer wave rate. Non-limiting examples of a bioimpedance sensor and a physiological characteristic determinator are described in U.S. patent application Ser. No. 13/802,319, filed on Mar. 13, 2013, which is herein incorporated herein by reference. Further, multiple sensor terminals 108 and 109 can contact a common portion of ear 150 (e.g., two sensor terminals can extract a bioimpedance signal from concha 102 portion of ear 150). In other instances, one or more sensor terminals can extract a bioimpedance signal from two or more regions (e.g., an AC signal can be injected into cymba concha 102 portion of ear 150 and extracted from concha cymba or cavum region 104 of ear 150).

In some embodiments, physiological sensor 140 includes a piezoelectric sensing element as a sensor terminal 108. In this case, sensor terminal 108 can be configured to sense, for example, acoustic energy and to generate an electric signal indicative to the characteristics of the acoustic energy. Sensor terminal 108 (as well as other sensor terminals 109) can be positioned adjacent to a source of physiological signals, such as adjacent to a blood vessel. According to some embodiments, physiological sensor 140 is a piezoelectric sensor 170 (e.g., a portion of which is a piezoelectric transducer) configured to receive, for example, acoustic energy, and further configured to generate piezoelectric signals (e.g., electrical signals). In the example shown, piezoelectric sensor 130 is configured to receive an acoustic signal that includes, for example, heart-related signals. For example, an acoustic signal can propagate through at least human tissue as sound energy waveforms. Such sound energy signals can originate from either a heart beating (e.g., via a blood vessel) or blood pulsing through a blood vessel, or both. The energy propagating as an acoustic signal into a sensor terminal of piezoelectric sensor 140, which is converts the acoustic energy into piezoelectric signals transmitted to physiological characteristic determinator 170. Physiological characteristic determinator 170, which, in some examples, can be described as a physiological signal generator, is configured to detect and identify, for example, heartbeats. An example of a piezoelectric sensor that can be implemented is described in U.S. patent application Ser. No. 13/672,398, filed on Nov. 8, 2012, both of which are incorporated by reference. As used herein, the term tissue can refer to, at least in some examples, as skin, muscle, blood, or other tissue.

In some embodiments, physiological sensor 130 can implement a microphone to detect acoustic energy and sound waves. A microphone (not shown) configured to contact (or to be positioned adjacent to) the skin of the wearer, whereby the microphone is adapted to receive sound and acoustic energy generated by the wearer (e.g., the source of sounds associated with physiological information). The microphone can also be disposed at the ear as a sensor terminal 108 and/or any of sensor terminal 109 (e.g., when differentially sensing acoustic signals). According to some embodiments, the microphone can be implemented as a skin surface microphone (“SSM”), or a portion thereof, according to some embodiments. An SSM can be an acoustic microphone configured to enable it to respond to acoustic energy originating from human tissue rather than airborne acoustic sources. As such, an SSM facilitates relatively accurate detection of physiological signals through a medium for which the SSM can be adapted (e.g., relative to the acoustic impedance of human tissue). Examples of SSM structures in which piezoelectric sensors can be implemented (e.g., rather than a diaphragm) are described in U.S. patent application Ser. No. 11/199,856, filed on Aug. 8, 2005, and U.S. patent application Ser. No. 13/672,398, filed on Nov. 8, 2012, both of which are incorporated by reference. As used herein, the term human tissue can refer to, at least in some examples, as skin, muscle, blood, or other tissue. Note that signal 119 can represent a raw bioimpedance signal (e.g., an electrical signal) or a piezoelectric signal (e.g., an electrical signal) that embodies data describing the physiological characteristics (i.e., some processing may be performed to extract physiological signals at physiological characteristic determinator 170). Or, signal 119 can represent the physiological signals. Note that in some embodiments, physiological signals can be related to any physiological signals (e.g., need not be limited to heart-related signals). Further, physiological sensor 140 can include a wireless transceiver (“RF”) 141 configured to transmit and receive radio frequency signals for communication physiological information, among other things.

FIG. 1B is a diagram illustrating another example of an ear-related device/implement, according to some embodiments. Diagram 190 depicts an ear-related device/implement 110, one or more physiological sensor(s), a sensor controller 180, and a physiological characteristic determinator 170. In some examples, elements or components depicted in FIG. 1B can have similar or equivalent structures and/or functionalities to similarly-named and/or similarly-numbered elements or components that are depicted in FIG. 1A. One or more sensor terminals 108, 109, and 111 can be implemented to sense signals embodying or otherwise including information describing one or more physiological characteristics. The one or more sensor terminals 108, 109, and 111 can be configured to receive and/or transmit electrical energy signals (e.g., bioimpedance signals, galvanic skin response (“GSR”) signals, and the like), acoustic energy signals (e.g., energy related to sounds based on, for example, a heartbeat, or any sounds propagating through tissue), magnetic or electromagnetic signals, optical energy signals (e.g., one or more subset of light in any spectrum or at any wavelength, including reflected light to perform, for example, pulse oximetry, and other types of light, including reflected or emitted light), pressure and/or force-related signals (e.g., tactile-related signals, etc.), and the like.

One or more sensor terminals 108, 109, and 111 can be positioned at or adjacent to any of the regions shown in ear 192. In some cases, a sensor terminal may contact a portion of tissue of ear 192, or related thereto. For example, any of the sensor terminals 108, 109, and 111 can be positioned to sense physiological signals at or near an antihelix 107 portion of ear 151 (e.g., interior surface portions, such as adjacent concha 102), and a portion of region 154 associated with concha 102 to detect, for example, bioimpedance-related, pressure-related, galvanic-related, and/or acoustic-related signals, among others. Portion 152 of blood vessel 151b or the adjacent skin can be a source of such signals, according to some examples. Further, any of the sensor terminals 108, 109, and 111 can be positioned at (e.g., in contact with) or adjacent to targus 103, including at or near exterior portion 105 or an interior portion (not shown).

In some other cases, a sensor terminal may be positioned near a portion of tissue of ear 192, or related thereto. For example, any of sensor terminals 108, 109, and 111 can be implemented to receive physiological signals from, or in association with (e.g., via), tissue area 101a, which is behind ear 192, tissue area 101b, or any other tissue area. Such sensor terminals can also be configured to receive physiological signals from blood vessels, such as blood vessel 151a or blood vessel 151b. Sensor terminals can be used to detect, for example, bioimpedance-related, pressure-related, galvanic-related, and/or acoustic-related signals, etc. in association with tissue areas 101a and 101b and blood vessels 151a and 151b.

Physiological sensor 140 of diagram 190 is configured to generate one or more physiological signals and can be used to derive physiological signals, such as heart rate, respiration, GSR, and other detectable physiological characteristics, for example, from the sensor terminals. Any ear-related device can include a physiological sensor 140 and a physiological characteristic determinator 170, which can be implemented as a physiological signal generator in some embodiments. Physiological sensor 140 can be configured to sense signals, such as physiological signals, associated with a physiological characteristic.

Ear-related device/implement 110 can coupled to or can include a physiological sensor 140 and/or a physiological characteristic determinator 170. Physiological sensor 140 is configured to receive the sensed signals from one or more of the sensor terminal 108, as well as from any of sensor terminals 109 and 111. In one embodiment, physiological sensor 140 includes a bioimpedance sensor 120, a piezoelectric sensor 130, a force-sensing sensor 160 and one or more other sensors 165. In at least one embodiment, force-sensing sensor 160 can be configured to detect an applied pressure or force, as well as changes in pressure or force. In one example, sensor 160 can be implemented as a force-sensing resistor (“FSR”). For example, sensor 160 can sense fluctuations in pressure via surface portion 105 of targus 103 due to changes in blood volume in blood vessel 151b or in tissue dimension. In other implementations, force-sensing resistor can be implemented as a strain gauge to detect an applied strain or compression (e.g., to detect inflammation and other physiological characteristics). In yet other examples, force-sensing sensor 160 can be configured to detect any changes, deformations, contractions, expansions, or any movement (cyclical or otherwise) in physical structure of an organism, such as movement of tissue and the like.

One or more other sensors 165 can also include optical sensors, temperature sensors (e.g., skin temperature, core temperature, such as in an ear canal, ambient air temperature, and the like), motion sensors (e.g., one or more accelerometers, gyroscopic sensors, optical motion sensors (e.g., laser or LED motion detectors, such as used in optical mice), and the like), environmental sensors (e.g., gas sensors, chemical sensors, etc.), and other sensors. Sensors 165 can also include magnet-based motion sensors (e.g., detecting magnetic fields, or changes thereof, to detect motion), electromagnetic-based sensors, etc., as well as any sensor configured to detect or determine motion, such as motion sensors based on physiological characteristics (e.g., using electromyography (“EMG”) to determine existence and/or amounts of motion based on electrical signals generated by muscle cells), and the like. One or more other sensors 165 can further include a heat flux sensor that may include a transducer, or another element, to generate a physiological signal indicative of an amount of thermal energy (e.g., heat) radiating or passing through a surface portion of tissue. In some embodiments, one or more portions of sensors 120, 130, 160, and 165 can be implemented as one or more portions of a sensor terminal.

Physiological characteristic determinator 170 is configured to receive one or more signals including physiological information (and other sensor-obtained information) via path 119, and is further configured to process (e.g., digitally) the signal data including one or more physiological characteristics to derive physiological signals, such as either a heart rate (“HR”) signal or a respiration signal, or both, or any other physiological signal or characteristic. For example, physiological characteristic determinator 170 is configured to amplify and/or filter the physiological-related component signals (e.g., at different frequency ranges) to extract certain physiological signals. According to various embodiments, a heart rate signal can include (or can be based on) a pulse wave. A pulse wave includes systolic components based on an initial pulse wave portion generated by a contracting heart, and diastolic components based on a reflected wave portion generated by the reflection of the initial pulse wave portion from other limbs. In some examples, an HR signal can include or otherwise relate to an electrocardiogram (“ECG”) signal. Physiological characteristic determinator 170 is further configured to calculate other physiological characteristics based on the acquired one or more physiological characteristics.

Optionally, physiological characteristic determinator 170 can use other information to calculate or derive physiological characteristics. Examples of the other information include motion-related data, including the type of activity in which the user is engaged, such as running or sleep, location-related data, environmental-related data, such as temperature, atmospheric pressure, noise levels, etc., and any other type of sensor data, including stress-related levels and activity levels of the wearer. One example of physiological characteristic determinator 170, or a variant thereof, can include a physiological characteristic determinator described in U.S. patent application Ser. No. 13/802,319, filed on Mar. 13, 2013, entitled “Determining Physiological State(s) of an Organism Based on Data Sensed with Sensors in Motion,” which is incorporated herein for all purposes.

Physiological state determinator 172 is configured to receive various physiological characteristics signals and to determine a physiological state of a user. Physiological states include, but are not limited to, states of sleep, wakefulness, a deviation from a normative physiological state (i.e., an anomalous state), an activity, such as running/walking, and the like. Physiological state determinator 172 can include activity managers, according to some embodiments. Examples of activity-related managers can include a nutrition manager, a sleep manager, an activity manager, a sedentary activity manager, and the like, examples of which can be found in U.S. patent application Ser. No. 13/433,204, filed on Mar. 28, 2012 having Attorney Docket No. ALI-013CIP1; U.S. patent application Ser. No. 13/433,208, filed Mar. 28, 2012 having Attorney Docket No. ALI-013CIP2; U.S. patent application Ser. No. 13/433,208, filed Mar. 28, 2012 having Attorney Docket No. ALI-013CIP3; U.S. patent application Ser. No. 13/454,040, filed Apr. 23, 2012 having Attorney Docket No. ALI-013CIP1CIP1; U.S. patent application Ser. No. 13/627,997, filed Sep. 26, 2012 having Attorney Docket No. ALI-100; all of which are incorporated herein by reference for all purposes.

In some example, physiological state determinator 172 can derive a physiological state or condition derived from one or more physiological characteristics signals based on the sensors. Examples of such physiological states or conditions include caloric intake and expenditure (e.g., a metabolism), a value for Metabolic Equivalent of Task (“METS”) or metabolism equivalent, and the like. Physiological state determinator 172 can include physiological characteristic determinator 170, or can be separate from physiological characteristic determinator 170, according to various examples. In some examples, physiological state determinator 172 can generate a notification signal (as a vibratory activation signal) to cause a vibratory energy source (e.g., mechanical motor as a vibrator), which is not shown, to impart vibration through a housing of ear device 110 unto a user, responsive to the vibratory activation signal, to indicate the presence of the sleep-related condition

Sensor controller 180 is configured to control functionality of one or more sensors, as well as interpretation of one or more physiological signals. In some embodiments, sensor controller 180 can operate to correct or modify a physiological signal based on other information, such as other physiological signals or conditions, or any environmental conditions. In a specific embodiment, sensor controller 180 is configured to obtain multiple physiological signals based on different sensors. Sensor controller 180 can correlate the multiple signals to, among other things, align them temporally. Sensor controller 180 can validate each of the multiple signals and, for example, can generate a correlated signal for representing or determining a physiological characteristic, such as heart rate. In some examples, without limitation, a correlated signal can be a composite of the multiple signals to ensure, for instance, accuracy in measuring the physiological characteristic. Further, sensor controller 180 can determine whether one of the multiple sensor signals are out-of-tolerance and can exclude the use of that signal.

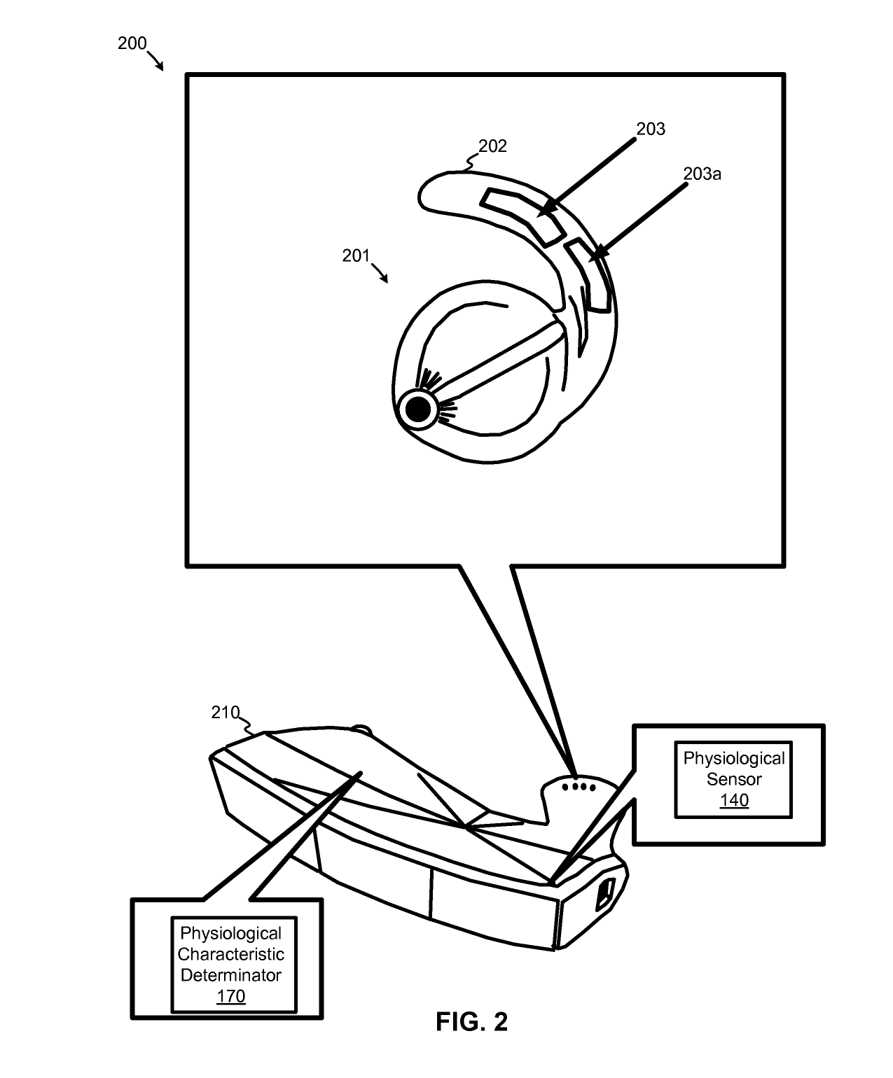

FIGS. 2 to 8 depict several examples and are not intend to be limiting. Various embodiments are broader than as described therein.

FIG. 2 depicts an ear-related device/implement configured to receive signals describing physiological characteristics, according to some embodiments. Diagram 200 depicts ear-related device/implement 210 including an earbud 201 having an extension structure 202 (e.g., a portion of a C-type earbud, such as those manufactured by Jawbone®) that includes one or more sensor terminals 203. In some examples, sensor terminals 203 are conductive and can be configured to apply and/or receive a bioimpedance signal. Such a signal can be received by ear-related device/implement 210 which includes at least physiological sensor 140. In some examples, sensor terminal 203 can be a piezoelectric transducer or related structures. Thus, physiological sensor 140 can be a bioimpedance sensor or an acoustic sensor, such as a piezoelectric sensor. In some examples of physiological characteristic determinator 170 can be disposed in ear-related device/implement 210, but can also be disposed in any other device, in communication with ear-related device/implement 210, such as a mobile device or phone. In some examples, extension structure 202 is configured to apply a spring-like force to a cymba concha so that sensor terminal 203 is in contact with tissue. In some cases extension structure 202 is configured to minimize vibrations (and noise associated therewith). Therefore, extension structure 202 can enhance signal quality and integrity of a sensed signal (e.g., improving a signal-to-noise ratio). According to some embodiments, one or more additional sensor terminals 203a can be implemented to implement multiple sensors (e.g., multiple FSRs), as well as bioimpedance sensory signals or other signals.

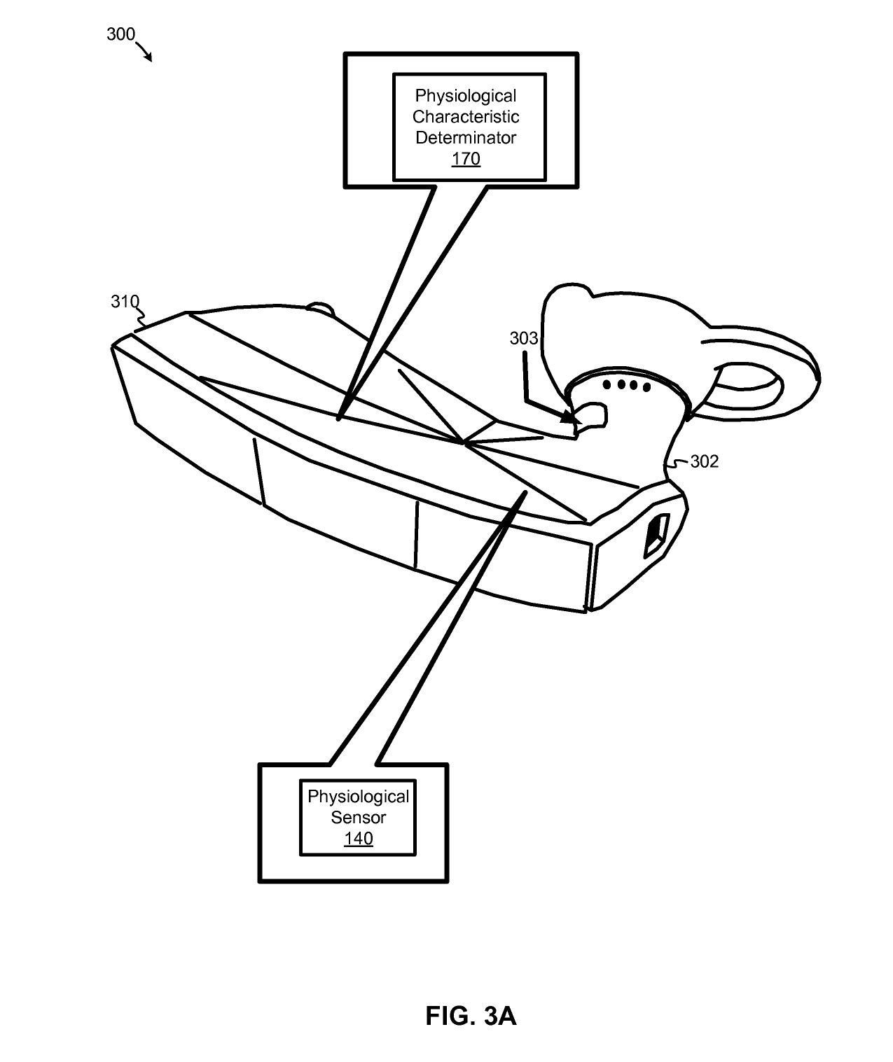

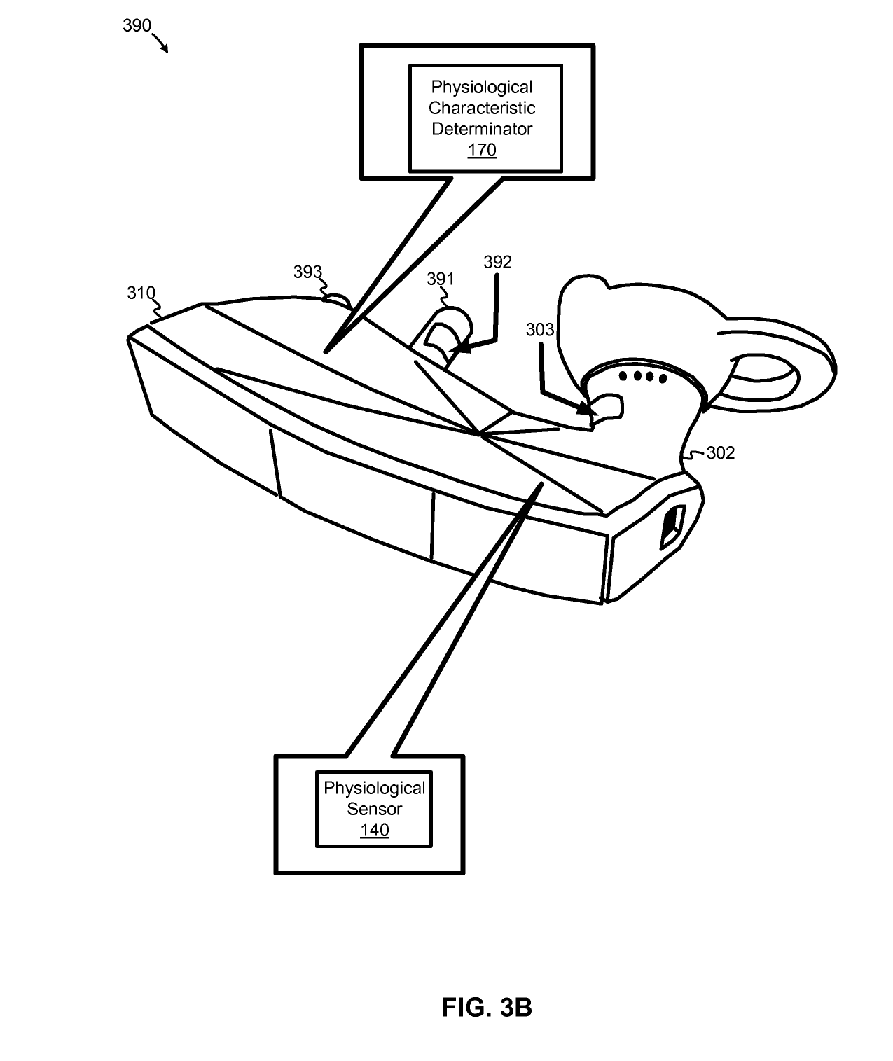

FIG. 3A depicts another example of an ear-related device/implement configured to provide for sensor terminals to sense physiological characteristics, according to some embodiments. Diagram 300 depicts ear-related device/implement 310 including a neck portion 302 that can include one or more sensor terminals 303. In some examples, sensor terminals 303 are conductive and are configured to apply and/or receive a bioimpedance signal. Such a signal can be received by ear-related device/implement 210 which includes at least physiological sensor 140. In some examples, sensor terminal 303 can be a piezoelectric transducer or related structures. Thus, physiological sensor 140 can be a bioimpedance sensor or an acoustic sensor, such as a piezoelectric sensor. In some examples of physiological characteristic determinator 170 can be disposed in ear-related device/implement 310, but can also be disposed in any other device, in communication with ear-related device/implement 310, such as a mobile device or phone (not shown). In some examples, neck portion 302 can be configured to apply a force to a portion of a targus portion (e.g., adjacent to the ear canal) inside of an ear so that sensor terminal 303 is in contact with tissue.

FIG. 3B depicts a variation of yet another example of an ear-related device/implement configured to provide for sensor terminals to sense physiological characteristics, according to some embodiments. In some examples, elements or components depicted in FIG. 3B can have similar or equivalent structures and/or functionalities to similarly-named and/or similarly-numbered elements or components that are depicted in FIG. 3A. Diagram 390 depicts an extension structure 391 that includes a sensor terminal 392. Sensor terminals 303 and 392 can be implemented as similar or different types of sensors. In at least one case, sensor terminals 303 and 392 can be implemented as force-sensing resistors. Further to diagram 390, structure 393 can be implemented as an SSM that can be implemented to enhance voice activity detection (“VAD”) and to detect acoustic energy signals emanating from blood vessels (e.g., heartbeat sounds). According to various embodiments, sensor terminals 303 and 392 can be implemented as any type of sensors, such as a bioimpedance-based sensor, an optical sensor (e.g., pulse oximetry sensor), or any other type of sensor.

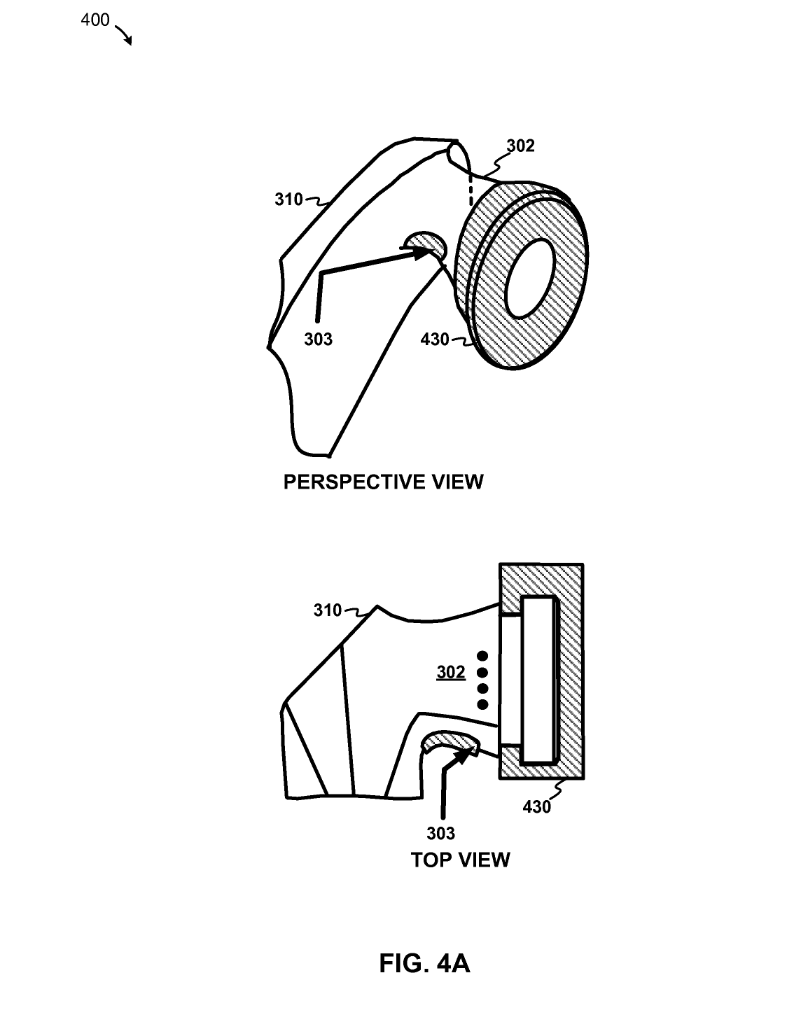

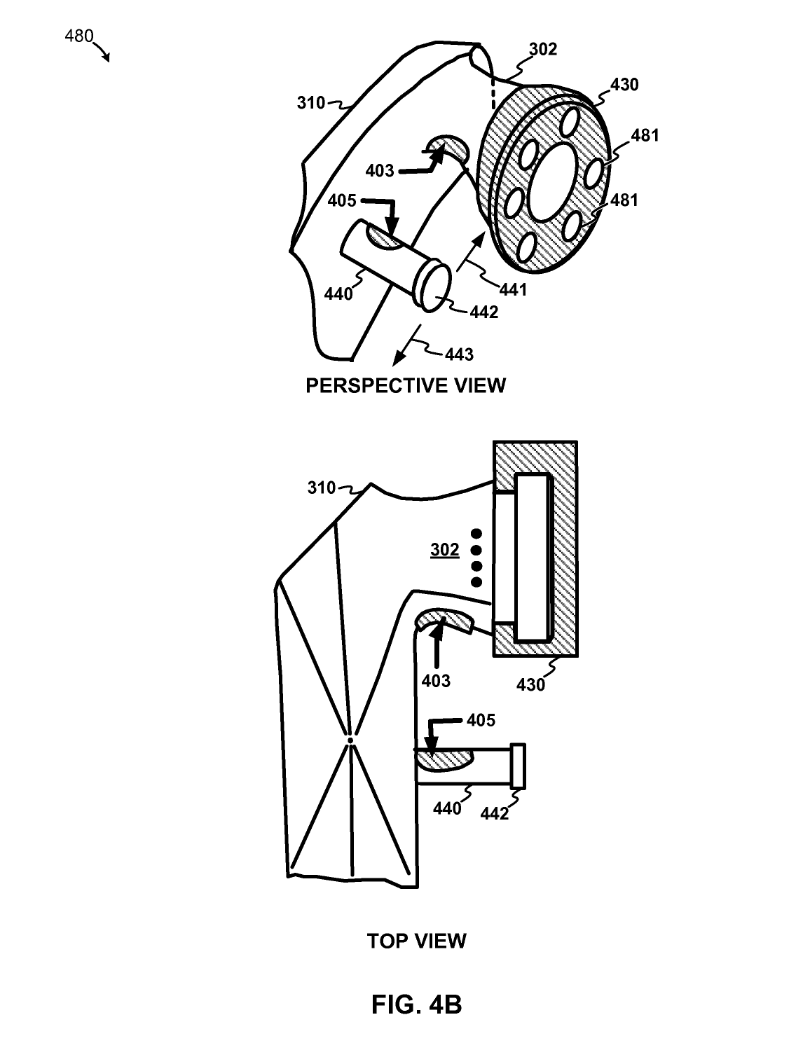

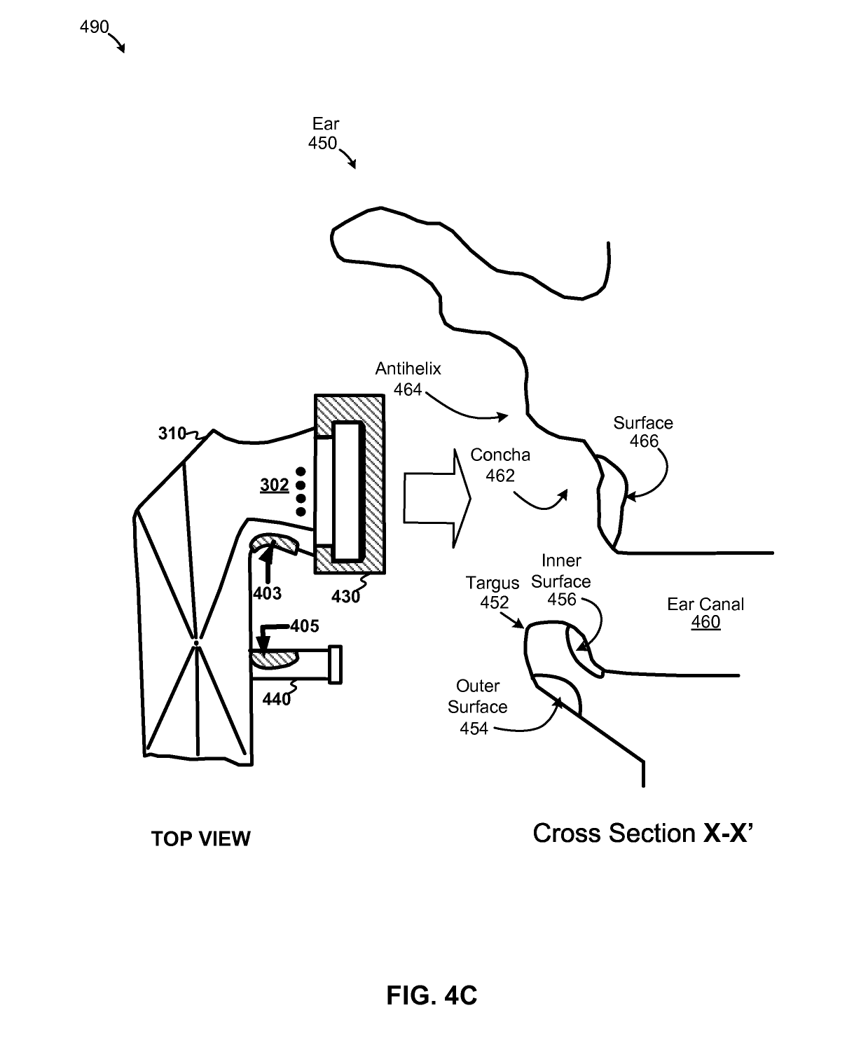

FIG. 4A depicts perspective and top views of the ear-related device/implement shown in FIG. 3A, according to some examples. Diagram 400 includes a perspective view and a top view. The perspective view depicts a sensor terminal 303 co-located on neck 302, whereby an earbud 430 is configured to contact portions of an ear canal to establish relatively firm contact between source terminal 303 and the tissue of the targus. The top view depicts the positioning of source terminal 303 on neck 302, along with earbud 430. Note that multiple source terminals 303 can be implemented at different portions of 302 to contact the targus or any other ear portion at multiple points.

FIG. 4B depicts perspective and top views of yet another example of an ear-related device/implement shown in FIG. 4A, according to some examples. In some examples, elements or components depicted in FIGS. 4B and 4C can have similar or equivalent structures and/or functionalities to similarly-named and/or similarly-numbered elements or components that are depicted in FIG. 4A. Diagram 480 of FIG. 4B includes a perspective view and a top view. As shown, device 310 can include an extension structure 440, which can include either a first sensor terminal 405 or a second sensor terminal 442, or both. In one embodiment, extension structure 440 is semi-rigid in its construction such that the tip of extension structure (e.g., at which sensor terminal 442 is disposed) can be displaced and resiliently return (or try to return) to its initial orientation. For example, the tip can displace in direction 443 when a user is placing device 310 at or adjacent to an ear (e.g., an insertion force can cause extension structure 440 to bend toward direction 443), and the tip can return to its original position to secure the tip against a tissue surface (e.g., under an influence of a spring-like force to return the tip toward direction 441). Thus, sensor terminal 405 can contact (e.g., firmly contact) a portion of tissue to receive physiological signals. In one implementation, one or both of sensor terminals 403 and 405 are force-sensing resistor sensors. Or, sensor terminals 403 and 405 can be optical sensors. Alternatively, sensor terminals 403 and 405 can be implemented with a bioimpedance sensor, or can be implemented in different sensors.

In various cases, sensor terminal 442 is optional and need not be implemented. Sensor terminal, 442, can be implemented as an SSM to receive acoustic energy signals. Further, earbud 430 can include one or more sensor terminals for implementing one or more of the following: bioimpedance-based sensing, resistive-based sensing (e.g., FSR), galvanic sensing, acoustic sensing, and the like. Sensor terminals 481 can be disposed adjacent to a concha portion of an ear (e.g., to contact tissue of concha).

FIG. 4C depicts an example of the coupling of an ear-related device shown in FIG. 4B to an ear, according to some examples. Diagram 490 depicts a device 310 and an ear 450. In this diagram, ear 450 is depicted as a cross-sectioned view X-X′ of ear 192 of FIG. 1B. Referring to FIG. 4C, an example of ear 450 includes an antihelix portion 464, a concha portion 42 including a surface 466 (e.g., surface of tissue in the concha), an ear canal 460, and a targus portion 452 including an inner surface 456 and an outer surface 454. Sensor terminal 403 is configured to engage or otherwise sense physiological signals in association with inner surface 456, whereas sensor terminal 405 is configured to engage or otherwise sense physiological signals in association with outer surface 454. In some embodiments, sensor terminals 403 and 405 are configured as force-sensing resistors (or portions thereof) to detect changes in pressure to determine a physiological signal. For example, as blood vessels expand with pulsating blood volume, pressure is exerted via tissue (e.g., an epidermis) to either sensor terminals 403 or 405, or both. Pressure is relieved as blood flows out from the blood vessels. Signals representing blood flow-induced pressure can be correlated and/or sensor terminals 403 and 405 can be correlated to determine whether one or both can be used or discarded, or whether one ought to be favored over the other. Note that sensor terminals 403 and 405 are not limited to force-sensing related sensors, but can be implemented as electrodes for a bioimpedance sensor. Or, sensor terminals 403 and 405 can represent an optical signal generator and receiver to implement pulse oximetry. In some cases, sensor terminals 403 and 405 can represent acoustic pickups for receiving acoustic energy. Moreover, sensor terminals 403 and 405 can include temperature sensors or any other type of sensor.

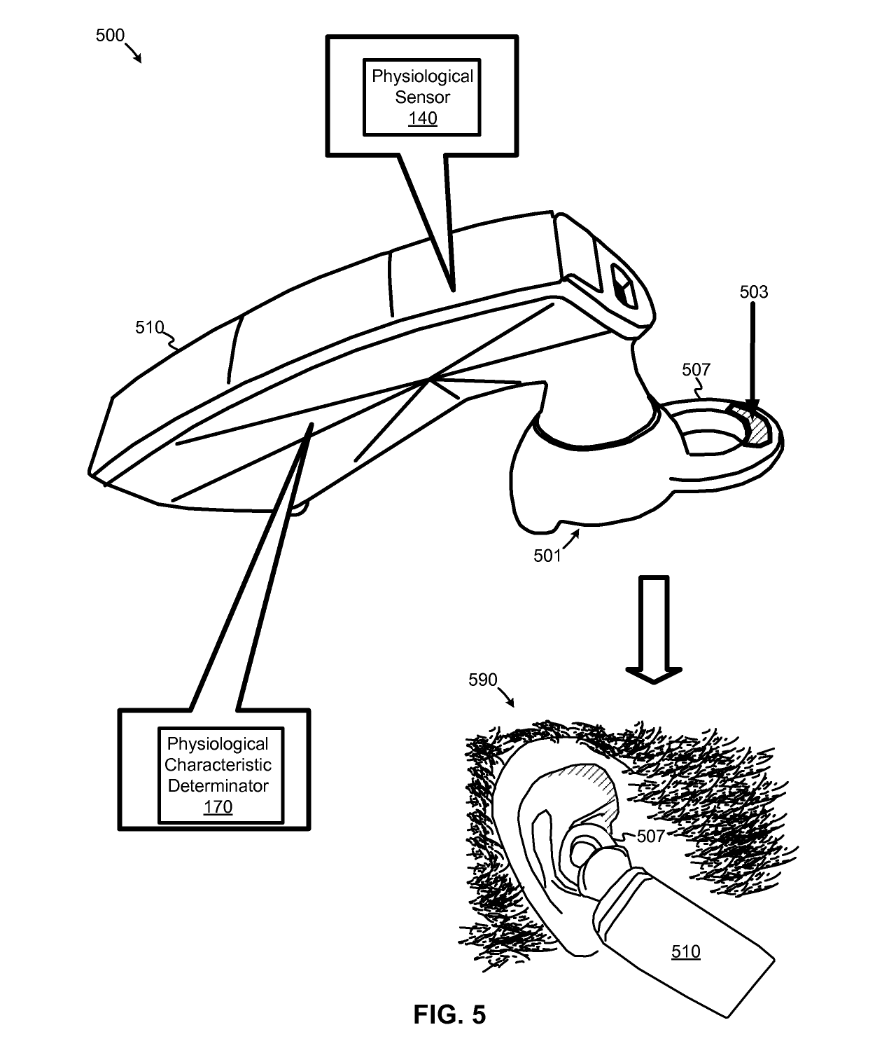

FIG. 5 depicts another example of an ear-related device/implement configured to provide for sensor terminals to sense physiological characteristics, according to some embodiments. Diagram 500 depicts ear-related device/implement 510 including an earbud 501 (e.g., a loop-spout bud) that can include one or more sensor terminals 503 disposed on or at loop portion 507. In some examples, sensor terminals 503 can be conductive and can be configured to apply and/or receive a bioimpedance signal. Such a signal can be received by ear-related device/implement 510 which includes at least physiological sensor 140. In some examples, sensor terminal 503 can be a piezoelectric transducer or related structures. Thus, physiological sensor 140 can be a bioimpedance sensor or an acoustic sensor, such as a piezoelectric sensor. In some examples of physiological characteristic determinator 170 can be disposed in ear-related device/implement 510, but can also be disposed in any other device, in communication with ear-related device/implement 510, such as a mobile device or phone (not shown). In some examples, loop portion 507 is inserted within an ear, as shown in diagram 590, whereby sensor terminal 503 can be positioned adjacent to or in contact with the concha cavum or the back of the concha. The loop portion 507 provides, at least in one example, a horizontal reaction force via the back of the concha, which can bend loop portion 507.

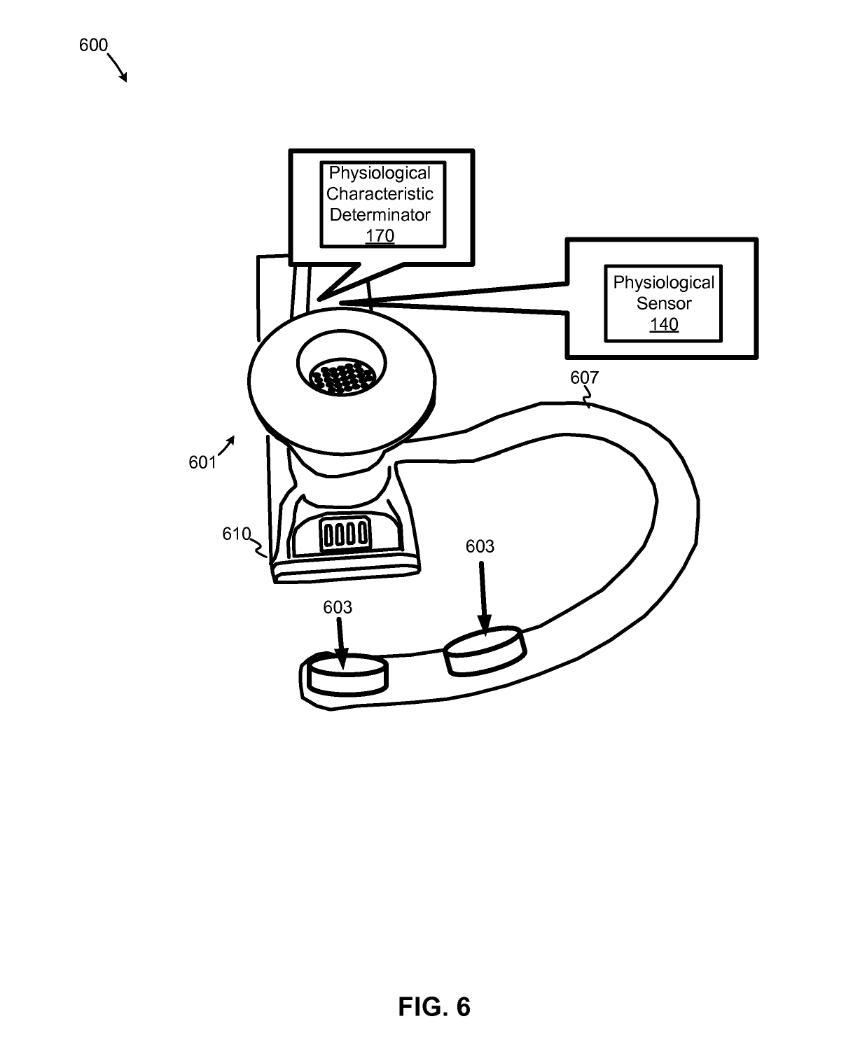

FIG. 6 depicts another example of an ear-related device/implement configured to provide for sensor terminals to sense physiological characteristics, according to some embodiments. Diagram 600 depicts ear-related device/implement 610 including an earbud 601 that can include one or more sensor terminals 603 disposed on or at a portion of an ear loop 607. In some examples, sensor terminals 603 are conductive and are configured to apply and/or receive a bioimpedance signal and/or a galvanic skin response (“GSR”) signal. Such a signal can be received by ear-related device/implement 610 which includes at least physiological sensor 140. In some examples, sensor terminal 603 can be a piezoelectric transducer or related structures. Also, sensor terminal 603 can be an SSM (or any type of acoustic sensor), or a force-sensing resistor-based sensor (or any type of pressure-related sensor). Thus, physiological sensor 140 can be a bioimpedance sensor or an acoustic sensor, such as a piezoelectric sensor. In some examples of physiological characteristic determinator 170 can be disposed in ear-related device/implement 610, but can also be disposed in any other device, in communication with ear-related device/implement 610, such as a mobile device or phone (not shown). In some examples, the portion of ear loop 607 is inserted behind an ear, whereby one or more sensor terminal 603s can be positioned adjacent to or in contact with tissue behind the ear. The loop portion 607 provides, at least in one example, a force via ear loop 607 to apply sensor terminals 603 to tissue.

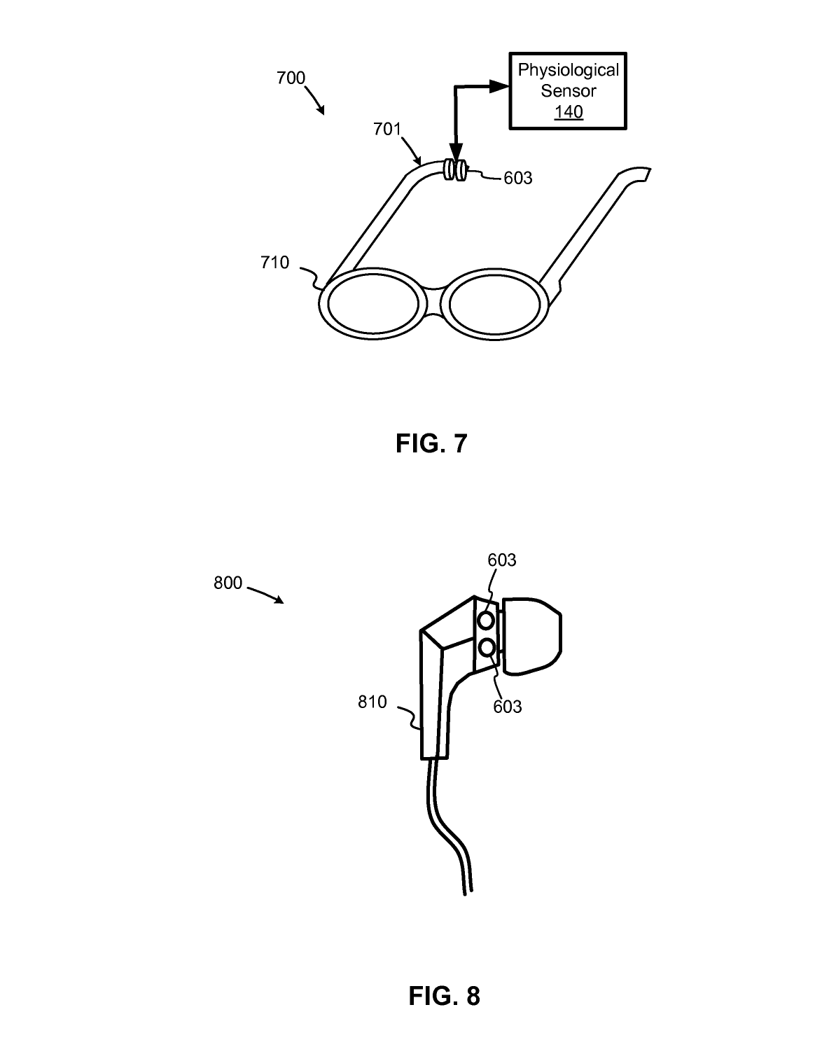

FIG. 7 depicts another example of an ear-related device/implement configured to provide sensor terminals to sense physiological characteristics, according to some embodiments. Diagram 700 depicts ear-related device/implement 610 as an implement (e.g., eyewear) including sensor terminals 603 disposed on or adjacent a temple tip 701 of eyewear 710.

FIG. 8 depicts yet another example of an ear-related device/implement configured to provide sensor terminals to sense physiological characteristics, according some embodiments. Diagram 800 depicts an earbud 810 configured to be inserted into an ear canal for providing audio. Earbud 810 can include sensor terminal 603 that are configured to contact tissues of the ear, such as at the ear canal. Therefore, earbud 810 can be used for sensing physiological characteristics, according to various embodiments.

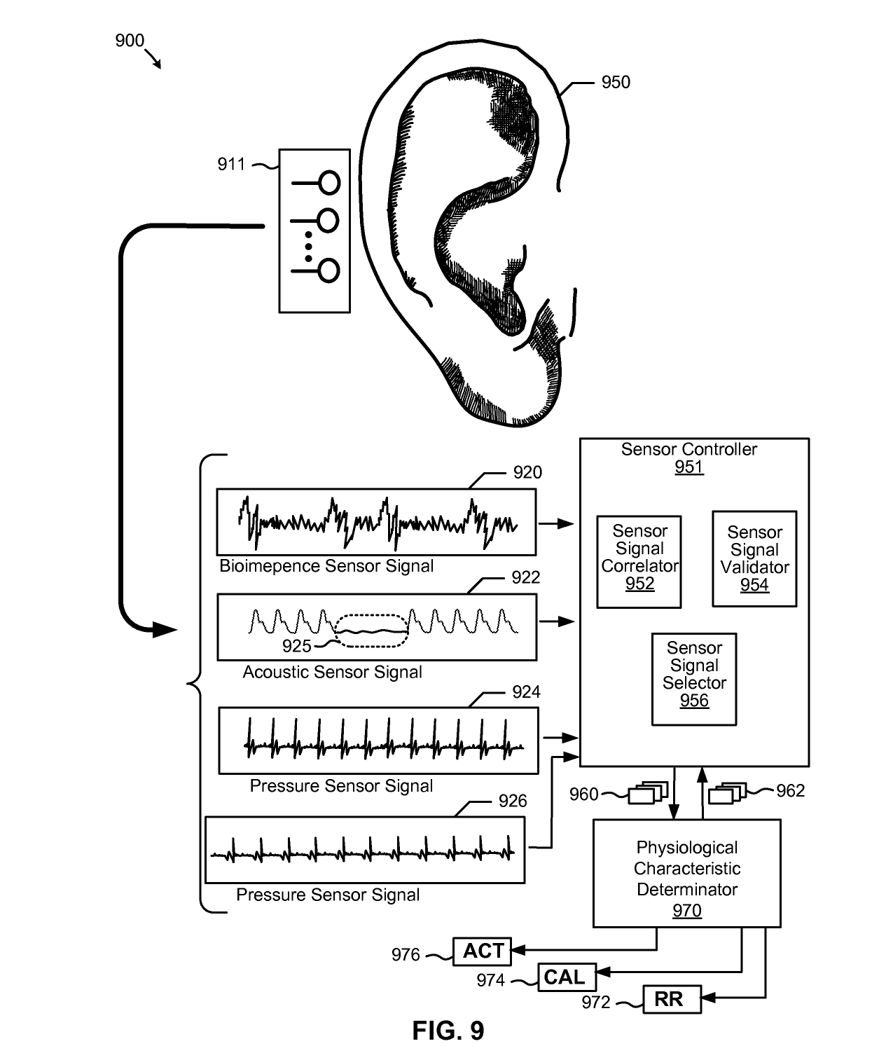

FIG. 9 is a diagram depicting a sensor controller, according to some examples. Diagram 900 includes a sensor controller configured to receive sensor signals from one or more sensor terminals 911 positioned at or adjacent an ear 950. Sensor controller 951 is configured to receive signals from one or more sensors, such as signals 920, 922, 924, and 926. In the example shown, signal 920 is depicted as an example of a bioimpedance sensor signal, signal 922 is depicted as an example of an acoustic sensor signal, signal 924 is depicted as an example of a pressure sensor signal (e.g., a force-sensing resistance signal), and signal 926 is depicted as another example of a pressure sensor signal. In some embodiments, sensor controller 951 can operate to correct or modify a physiological signal based on other information, such as other physiological signals or conditions, or an environmental condition. In a specific embodiment, sensor controller 951 is configured to obtain multiple physiological signals based on different sensors originating at or adjacent an ear (e.g., in an ear-related device).

Sensor controller 951 includes a sensor signal correlator 952, a sensor signal validator 954, and a sensor signal selector 956. Sensor signal correlator 952 is configured to correlate the multiple signals to, among other things, align them temporally. For example, sensor signal correlator 952 can operate to correlate signals 920 and 922 from different sensors to determine whether a common physiological signal, for example representing a heartbeat, can be correlated between two sensors. If so, signals 920 and 922 can be used to form a correlated signal, such as a composite of signals 920 and 922. Note, too, that sensor signal correlator 952 can be configured to correlate similar signals 924 and 924, both of which can represent force-sensing sensor signals (e.g., originating from sensor terminals 403 and 405 of FIG. 4C).

Sensor signal validator 954 can be configured to validate the correlated signals. Further, sensor signal validator 954 can invalidate a sensor signal if the corresponding sensor should become an unreliable source of physiological signals. For example, consider that signals 920 and 922 are used to derive a heart rate signal. During a time interval 925, portion of signal 922 is unreliable and thus can be invalidated. Sensor signal selector 956 selects signal 920 as a heart rate signal. If another sensor signal, such as signal 924, is available, sensor signal selector 956 can use signals 920 and 924 to derive a heart rate signal.

Physiological characteristic determinator 970 (and/or physiological state determinator, which is not shown) can be configured to receive one or more physiological sensor signals 960 to determine a physiological characteristic, such as respiration rate (“RR”) 972 or a heart rate. Physiological characteristic determinator 970 can also determine an activity and associated activity data (“ACT”) 976, such as a number of steps or an instance at which a foot strike is detected. Physiological characteristic determinator 970 can also determine whether a user is sleeping based on activity data 976. Further, physiological characteristic determinator 970 can determine data representing caloric expenditure (“CAL”) 974, an indication of a metabolism level, and the like. Note, too, that in some examples, physiological characteristic determinator 970 can transmit data 962 indicating that a sensor-based signal is out-of-tolerance based on a determine physiological characteristic value. Sensor controller 951 can use this information to select an appropriate sensor signal.

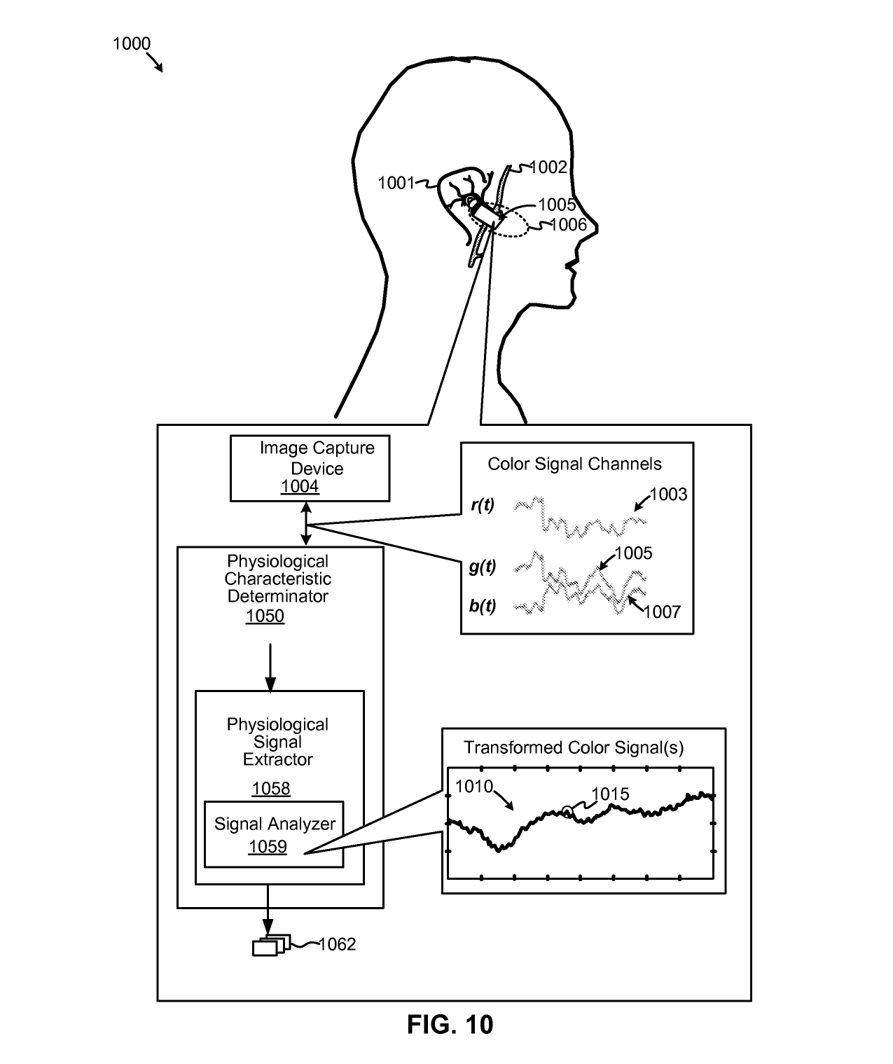

FIG. 10 is a diagram depicting an ear device implementing an optical sensor, according to some examples. Diagram 1000 depicts an ear-related device, such as headset 1005, being associated with ear 1001 of a user. In some embodiments, device 1005 includes an optical sensor, such as an image capture device 1004, that is configured to capture light. Examples of capture light includes light reflect off surface portion 1006, which is located over and/or adjacent to blood vessel 1002. The reflected light can originate from tissue, such as a surface portion 1006 of the face of an organism or person, whereby the reflected light (e.g., any spectrum including visible light) can include physiological information based on face flushing or flushes (e.g., can include plethysmographic signal information). A face flushing state can be accompanied by an enhanced blood volume, at least in some cases, at or near the surface of tissue, whereby a non-flushed state indicates a relatively lower blood volume. According to some embodiments, the transitions between face flushing states and non-flushing states can be due to pulsations of blood (i.e., heart beats), from which heart-related information (e.g., heart beat timings, heart rate, etc.) may be acquired via the reflected light (or other electromagnetic waveforms).

In the example shown, device 1005 can include an image capture device 1004, such as a camera, that is configured to receive light. Device 1005 also can include a physiological characteristic determinator 1050 configured to generate data 1062 representing a heart rate. As shown, physiological characteristic determinator 1050 can include a physiological signal extractor 1058 identifies a first subset of frequencies (e.g., a range of frequencies, including a single frequency) constituting green visible light, a second subset of frequencies constituting red visible light, and a third subset of frequencies constituting blue visible light. Other frequencies and wavelengths are possible, including those outside the visible spectrum. As shown, physiological characteristic determinator 1050 receives red visible light via a red color channel 1003, green visible light via a green color channel 1005, and blue visible light via a blue color channel 1007, any of which can include pixel values or other color-related signal values. In some cases, physiological signal extractor 1058 apply wavelet transforms, such as a continuous wavelet transform (“CWT”), data representing color-related signal values of, for example, a linearly-combined color channel signal to form a transformed color signal 1010. Signal analyzer 1059 can be implemented to identify portions 1015 of transformed color signal to detect heart beats and to estimate a heart rate from video in real-time. An example of a physiological signal extractor is described in U.S. patent application Ser. No. 13/967,317, which is incorporated herein by reference.

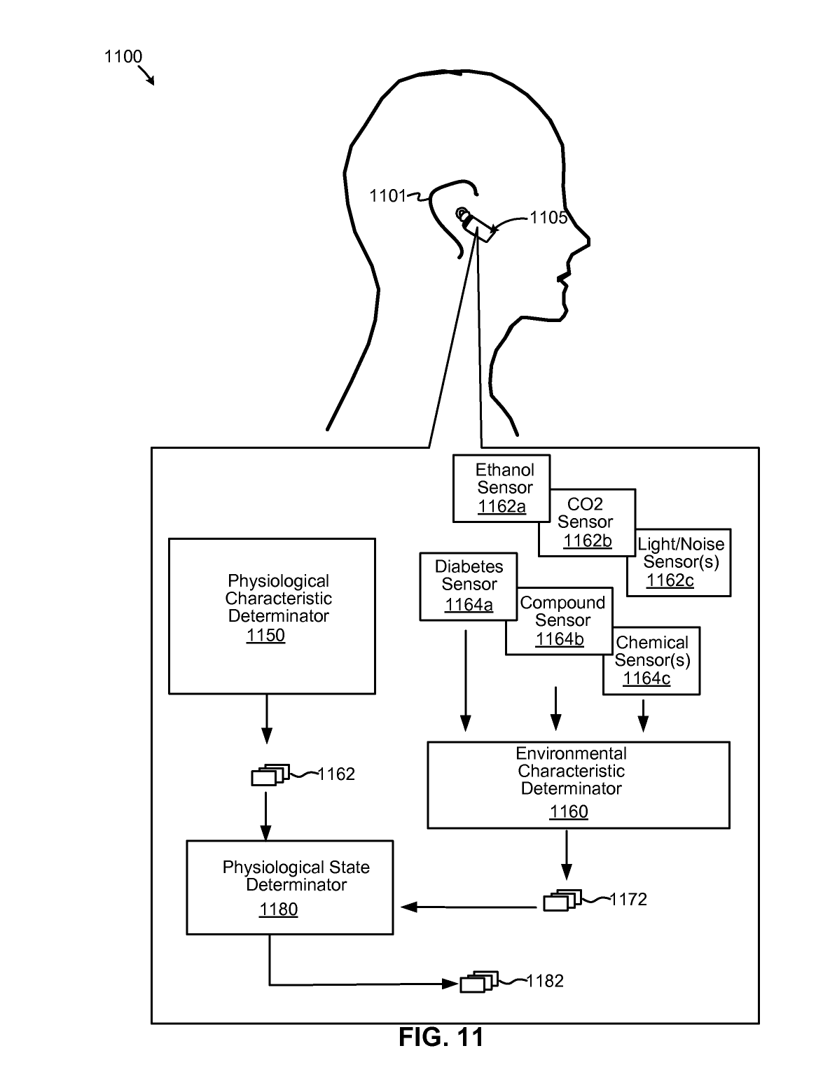

FIG. 11 is a diagram depicting an ear device implementing an environmental sensor, according to some examples. Diagram 1100 depicts an ear-related device, such as headset 1105, being associated with ear 1101 of a user. In some embodiments, device 1105 includes one or more environmental sensors 1162a-c and 1164a-c, or the like. Further, device 1005 may include a physiological characteristic determinator 1150, an environmental characteristic determinator 1160, and a physiological state determinator 1180. As described herein, physiological characteristic determinator 1150 can determine data 1162 representing sensed physiological characteristics, such as heart rate. Environmental characteristic determinator 1160 can determine data 1172 representing sensed characteristics of the environment. Examples of such characteristics can be sensed by environmental sensors 1162a-c, which can sense an amount of alcohol consumed (e.g., concentration of ethanol in exhaled water vapor), an amount of CO2 (or other gases) adjacent a face of a user (e.g., exhaled CO2), an amount of ambient light and/or noise, and the like. In other examples, device 1105 can include a diabetes sensor 1164a configured to detect, for example, an amount of one or more gases, such as acetone, to determine a condition associated with diabetes. Compound sensor 1164b can be configured to detect any number of compounds composed of multiple chemicals (e.g., one or more volatile organic compounds, or “VOCs”), and chemical sensor 1164c can be configured to detect a specific chemical. Other environmental sensors are also possible.

Physiological data 1162 and environmental data 1172 can be transmitted to physiological state determinator 1180 to determine a state of a user (e.g., an activity, such as sleep, or a condition of a user, such as pre-epileptic seizing, and the like), and can determine how the user's state is affected by the environment. In some instances, physiological state determinator 1180 can evaluate data from ethanol sensor 1162a and physiological data 1162, including GSR, HR, etc., to determine whether a person is intoxicated or has exceeded consuming a predetermined level of alcohol. Further, an amount of activity can be determined by evaluating an amount of CO2 sensed, for example, during strenuous aerobic exercise. Light and noise data can be used determine whether a person's sleep is affected by such environmental issues.

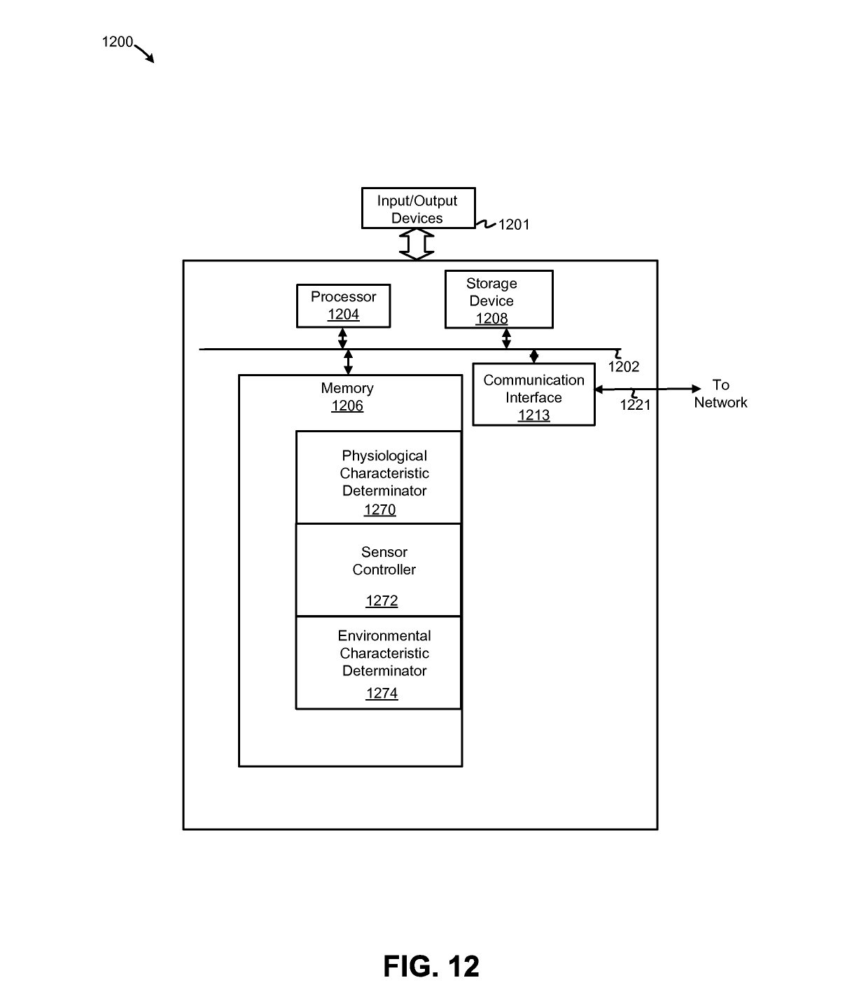

FIG. 12 illustrates an exemplary computing platform disposed in a device configured to provide physiological characteristics in accordance with various embodiments. In some examples, computing platform 1200 may be used to implement computer programs, applications, methods, processes, algorithms, or other software to perform the above-described techniques.

In some cases, computing platform can be disposed in an ear-related device/implement, a mobile computing device, or any other device.

Computing platform 1200 includes a bus 1202 or other communication mechanism for communicating information, which interconnects subsystems and devices, such as processor 1204, system memory 1206 (e.g., RAM, etc.), storage device 12012 (e.g., ROM, etc.), a communication interface 1213 (e.g., an Ethernet or wireless controller, a Bluetooth controller, etc.) to facilitate communications via a port on communication link 1221 to communicate, for example, with a computing device, including mobile computing and/or communication devices with processors. Processor 1204 can be implemented with one or more central processing units (“CPUs”), such as those manufactured by Intel® Corporation, or one or more virtual processors, as well as any combination of CPUs and virtual processors. Computing platform 1200 exchanges data representing inputs and outputs via input-and-output devices 1201, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 1200 performs specific operations by processor 1204 executing one or more sequences of one or more instructions stored in system memory 1206, and computing platform 1200 can be implemented in a client-server arrangement, peer-to-peer arrangement, or as any mobile computing device, including smart phones and the like. Such instructions or data may be read into system memory 1206 from another computer readable medium, such as storage device 1208. In some examples, hard-wired circuitry may be used in place of or in combination with software instructions for implementation. Instructions may be embedded in software or firmware. The term “computer readable medium” refers to any tangible medium that participates in providing instructions to processor 1204 for execution. Such a medium may take many forms, including but not limited to, non-volatile media and volatile media. Non-volatile media includes, for example, optical or magnetic disks and the like. Volatile media includes dynamic memory, such as system memory 1206.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1202 for transmitting a computer data signal.

In some examples, execution of the sequences of instructions may be performed by computing platform 1200. According to some examples, computing platform 1200 can be coupled by communication link 1221 (e.g., a wired network, such as LAN, PSTN, or any wireless network) to any other processor to perform the sequence of instructions in coordination with (or asynchronous to) one another. Computing platform 1200 may transmit and receive messages, data, and instructions, including program code (e.g., application code) through communication link 1221 and communication interface 1213. Received program code may be executed by processor 1204 as it is received, and/or stored in memory 1206 or other non-volatile storage for later execution.

In the example shown, system memory 1206 can include various modules that include executable instructions to implement functionalities described herein. In the example shown, system memory 1206 includes a physiological characteristic determinator 1270, a sensor controller 1272, and an environmental characteristic determinator 1274, one or more of which can be configured to provide or consume outputs to implement one or more functions described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. According to some embodiments, the term “module” can refer, for example, to an algorithm or a portion thereof, and/or logic implemented in either hardware circuitry or software, or a combination thereof. These can be varied and are not limited to the examples or descriptions provided.

In some embodiments, a physiological sensor and/or physiological characteristic determinator can be in communication (e.g., wired or wirelessly) with a mobile device, such as a mobile phone or computing device, or can be disposed therein. In some cases, a mobile device, or any networked computing device (not shown) in communication with a physiological sensor and/or physiological characteristic determinator, can provide at least some of the structures and/or functions of any of the features described herein. As depicted in FIG. 1A and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in any of the figure can represent one or more algorithms. Or, at least one of the elements can represent a portion of logic including a portion of hardware configured to provide constituent structures and/or functionalities.

For example, a physiological sensor and/or physiological characteristic determinator, or any of their one or more components can be implemented in one or more computing devices (i.e., any mobile computing device, such as a wearable device, an audio device (such as headphones or a headset) or mobile phone, whether worn or carried) that include one or more processors configured to execute one or more algorithms in memory. Thus, at least some of the elements in FIG. 1A (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, a physiological sensor and/or physiological characteristic determinator, including one or more components, can be implemented in one or more computing devices that include one or more circuits. Thus, at least one of the elements in FIG. 1A (or any subsequent figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A method comprising:
receiving one or more signals from one or more sensor terminals, the one or more sensor terminals being disposed to contact portions of tissue at or in an ear of an organism;
detecting an amount of pressure as a first signal associated with a sensor terminal of the one or more sensor terminals;
determining a physiological characteristic of the organism based on the amount of the pressure; and
generating data representing the physiological characteristic.

2. The method of claim 1, wherein detecting the amount of pressure comprises:
receiving a signal representing a value of a resistance responsive to the amount of pressure.

3. The method of claim 2, wherein determining the physiological characteristic comprises:
determining a heart-related characteristic.

4. The method of claim 2, further comprising:
implementing a force-sensing resistor to determine generate the value of the resistance responsive to the amount of pressure.

5. The method of claim 1, further comprising:
detecting a value associated with a second signal associated with the sensor terminal or another terminal of the one or more sensors terminals.

6. The method of claim 5, further comprising:
correlating the first signal and the second signal to form a correlated signal; and
implementing the correlated signal to form the data representing the physiological characteristic.

7. The method of claim 5, further comprising:
detecting an invalid portion of the first signal and the second signal; and
implementing the other of the first signal and the second signal to form the data representing the physiological characteristic.

8. The method of claim 1, further comprising:
causing positioning of the sensor terminal to contact an outer surface of tissue associated with a targus region of the ear; and
receiving the first signal from the outer surface.

9. The method of claim 1, further comprising:
causing positioning of the sensor terminal to contact an inner surface of tissue associated with a targus region of the ear; and
receiving the first signal from the inner surface.

10. The method of claim 1, wherein determining the physiological characteristic comprises:
determining a heart rate.

11. A wearable device comprising:
one or more sensor terminals;
one or more physiological sensors coupled to the one or more sensor terminals, the one or more physiological sensors configured to sense one or more signals originating at the one or more sensor terminals, at least a sensor terminal of the one or more sensor terminals comprising:
a pressure-sensitive terminal configured to detect a pressure exerted by a portion of tissue of an organism and generate a pressure signal representing a value of the pressure; and
a processor configured to receive the one or more signals, including the pressure signal, and further configured to cause generation of data representing a physiological characteristic of the organism.

12. The wearable device of claim 11, wherein the pressure-sensitive terminal comprises:
a force-sensing resistor (“FSR”).

13. The wearable device of claim 11, wherein the processor is further configured to cause generation of the data as a heart rate.

14. The wearable device of claim 11, further comprising:
an extension structure configured to position the one or more sensor terminals to contact portions of tissue at or in an ear of an organism, and is further configured to apply one or more forces to the one or more sensor terminals to maintain contact with the portions of tissue.

15. The wearable device of claim 14, wherein at least a portion of the extension structure includes the sensor terminal, the portion of the extension structure being oriented facilitate coupling between the sensor terminal and a surface of a targus region of an ear of the organism.

16. The wearable device of claim 11, further comprising:
a first physiological sensor including the pressure-sensitive terminal;
a second physiological sensor; and
sensor controller configured to select either the first physiological sensor or the second physiological sensor, or both, to receive the receive the one or more signals.

17. The wearable device of claim 16, wherein the second physiological sensor comprises:
a bioimpedance sensor.

18. The wearable device of claim 16, wherein the second physiological sensor comprises:
a piezoelectric sensor.

19. The wearable device of claim 16, wherein the second physiological sensor comprises:
a skin surface microphone (“SSM”).

20. The wearable device of claim 11, further comprising one or more of an image capture device and an environmental sensor.
